(19) United States
(12) Patent Application Publication
(10) Pub. No.: US 2025/0041038 A1
(43) Pub. Date: Feb. 6, 2025

(54) Canine Unicompartimental Elbow Humeral Implant

(71) Applicant: Arthrex, Inc., Naples, FL (US)

(72) Inventors: Christoph Chronz, Muchen (DE); Dejan Zolnir, Munchen (DE); Elvir Hadzihafizovic, Pastetten (DE)

(21) Appl. No.: 18/715,312

(22) PCT Filed: Dec. 2, 2022
(86) PCT No.: PCT/US2022/051609

§ 371 Date: May 31, 2024

Related U.S. Application Data
(60) Provisional application No. 63/265,199, filed on Dec. 10, 2021.

(51) Int. Cl. A61D 99/00; A61F 2/30; A61F 2/38

(52) U.S. Cl. CPC A61D 99/00; A61F 2002/307; A61F 2002/3822

(57) ABSTRACT

A humeral implant comprising: a base having a first end and a second end opposite the first end, wherein the base includes a sidewall coupling the first end to the second end; and an articulating surface positioned at the second end of the base, wherein the articulating surface includes a first edge and a second edge opposite the first edge, wherein a radius of curvature of the first edge is greater than a radius of curvature of the second edge, and wherein the second edge of the articulating surface extends away from the sidewall of the base such that a surface area of the articulating surface is greater than a surface area of the first end of the base.

### CROSS-REFERENCE TO RELATED APPLICATIONS

This application claims the benefit of priority to U.S. Provisional Application No. 63/265,199 entitled “Canine Unicompartimental Elbow Humeral Implant,” filed on Dec. 10, 2021, the contents of which are hereby incorporated by reference in their entirety.

### BACKGROUND

The medial compartment disease (MCD) is a frequent pathology in canine elbows. A less invasive treatment option comprises a partial joint resurfacing including humeral and ulnar implants, which obviates the need for osteotomy or joint replacement. An improved humeral implant for canine elbow reconstruction is needed which prevents lateral impingement and improved wear reduction of the ulnar implant.

### SUMMARY

The present disclosure provides a humeral implant and methods for reconstructive elbow surgery, with particular application to canine elbow surgery.

In an aspect, a humeral implant can include a base having a first end and a second end opposite the first end. The base includes a sidewall coupling the first end to the second end. The humeral implant can also include an articulating surface positioned at the second end of the base. The articulating surface includes a first edge and a second edge opposite the first edge, where a radius of curvature of the first edge is greater than a radius of curvature of the second edge. The second edge of the articulating surface extends away from the sidewall of the base such that a surface area of the articulating surface is greater than a surface area of the first end of the base.

In another aspect, methods can include forming a humeral trough in a humeral condyle, and inserting the humeral implant of the above aspect into the humeral trough.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a side view of an example humeral implant.

FIG. 2 is a top view of the example humeral implant of FIG. 1.

FIG. 3 illustrates a humeral trough in a humeral condyle.

FIG. 4 illustrates an example humeral implant positioned in the humeral trough of FIG. 3.

### DETAILED DESCRIPTION

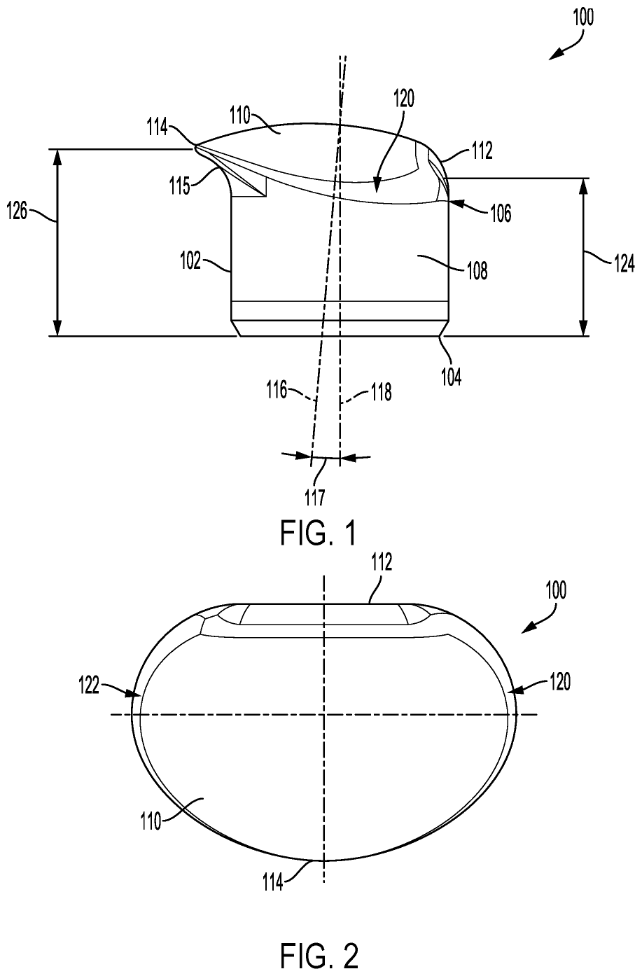

With reference to the Figures, FIGS. 1-2 illustrate a humeral implant 100. As shown in FIG. 1, the humeral implant 100 includes a base 102 having a first end 104 and a second end 106 opposite the first end 104. The base 102 includes a sidewall 108 coupling the first end 104 to the second end 106. The humeral implant 100 also includes an articulating surface 110 positioned at the second end 106 of the base 102. The articulating surface 110 includes a first edge 112 and a second edge 114 opposite the first edge 112. A radius of curvature of the first edge 112 is greater than a radius of curvature of the second edge 114. The second edge 114 of the articulating surface 110 extends away from the sidewall 108 of the base 102 such that a surface area of the articulating surface 110 is greater than a surface area of the first end 104 of the base 102.

In an example, as shown in FIG. 1, the first edge 112 of the articulating surface 110 is aligned with the sidewall 108 of the base 102. As further shown in FIG. 1, in an example an axis 116 perpendicular to a midpoint of the articulating surface 110 is offset 117 from a longitudinal axis 118 of the base 102 in a direction towards the second edge 114. In an example, the offset 117 ranges from about 3 degrees to about 7 degrees, from about 4 degrees to about 6 degrees, or about 5 degrees. Such an offset 117 is designed to match a surface of the humeral bone to smooth out the transition from the axial edge (first edge 112) to the abaxial edge (second edge 114).

In an example, a minimum radius of curvature of the first edge 112 ranges from about 1 mm to about 5 mm, and a minimum radius of curvature of the second edge 114 ranges from about 0.01 mm to about 3 mm. In an example, the first edge 112 may include two or more sections, each with a different radius of curvature. Such an example may help smooth out the first edge 112. In another example, the first edge 112 has a single section with a single radius of curvature. In an example, as shown in FIG. 1, the humeral implant 100 may include a curved surface 115 connecting the second edge 114 to the sidewall 108. The curved surface 115 may have a radius of curvature ranging from about 1 mm to about 3 mm. As shown in FIG. 2, in a top view the first edge 112 can be substantially straight, and from the top view the second edge 114 can be curved.

As shown in FIG. 2, in an example the articulating surface 110 can further include a first side edge 120 coupling the first edge 112 to the second edge 114 and a second side edge 122 opposite the first side edge 120 and coupling the first edge 112 to the second edge 114. In an example, the first side edge 120 and the second side edge 122 each have a variable radius of curvature that decreases from the first edge 112 to the second edge 114. As shown in FIGS. 1-2, in an example the articulating surface 110 can have a single line of symmetry. The base 102 can be oval in shape and therefore can have two lines of symmetry.

In an example, as shown in FIG. 1, a height 124 of the first edge 112 with respect to the first end 104 of the base 102 is less than a height 126 of the second edge 114 with respect to the first end 104 of the base 102. Such a height difference between the first edge 112 and the second edge 114 provides a spacer effect that helps to ensure that the articulating surface 110 of the humeral implant 100 is in constant contact with the ulnar component of the prosthetic elbow during the entire range of motion of the prosthetic elbow when in use.

In an example, the humeral implant 100 is formed of cobalt-chrome alloy and all surfaces of the humeral implant 100 (except the articulating surface 110) may be coated with CP titanium plasma spray. The humeral implant 100 may be also formed of a metal such as titanium, titanium alloy, stainless steel, or other materials that possess mechanical and physical properties suitable for bone repair. In an example, humeral implant 100 has dimensions of about 6 mm by about 10.5 mm with an implant height of about 6 mm, and may be formed of cobalt chrome. The humeral implant 100 may be provided in various sizes (for example, small, medium or large) and may be press fit into a humeral trough, as discussed in additional detail below.

In another example, the first end 104 of the base 102 may comprise a first material, and the second end 106 of the base 102 comprises a second material that is different than the first material. In an example, the first material is diffusion bonded to the second material.

In some examples, such as shown in any one of FIGS. 1-2, one or more components of the humeral implant 100 is made via an additive manufacturing process using an additive-manufacturing machine, such as stereolithography, multi-jet modeling, inkjet printing, selective laser sintering/melting, and fused filament fabrication, among other possibilities. Additive manufacturing enables one or more components of the humeral implant 100 and other physical objects to be created as intraconnected single-piece structure through the use of a layer-upon-layer generation process. Additive manufacturing involves depositing a physical object in one or more selected materials based on a design of the object. For example, additive manufacturing can generate one or more components of the humeral implant 100 using a Computer Aided Design (CAD) of the humeral implant 100 as instructions. As a result, changes to the design of the humeral implant 100 can be immediately carried out in subsequent physical creations of the humeral implant 100. This enables the components of the humeral implant 100 to be easily adjusted or scaled to fit different types of applications (e.g., for use with various types and sizes of canine elbow joints).

The layer-upon-layer process utilized in additive manufacturing can deposit one or more components of the humeral implant 100 with complex designs that might not be possible for devices assembled with subtractive manufacturing. In turn, the design of the humeral implant 100 can include aspects that aim to improve overall operation. For example, the design can incorporate physical elements that help redirect stresses in a desired manner that traditionally manufactured devices might not be able to replicate.

Additive manufacturing also enables depositing one or more components of the humeral implant 100 in a variety of materials using a multi-material additive-manufacturing process. In such an example, the majority of the humeral implant 100 may be made from a first material and the second end 106 of the base 102 and/or the articulating surface 110 may be made from a second material that is different than the first material. In another example, the entire humeral implant 100 is made from the same material. Other example material combinations are possible as well. Further, one or more components of the humeral implant 100 can have some layers that are created using a first type of material and other layers that are created using a second type of material.

Methods disclosed herein can be used with any of the embodiments of the humeral implant 100 as described herein.

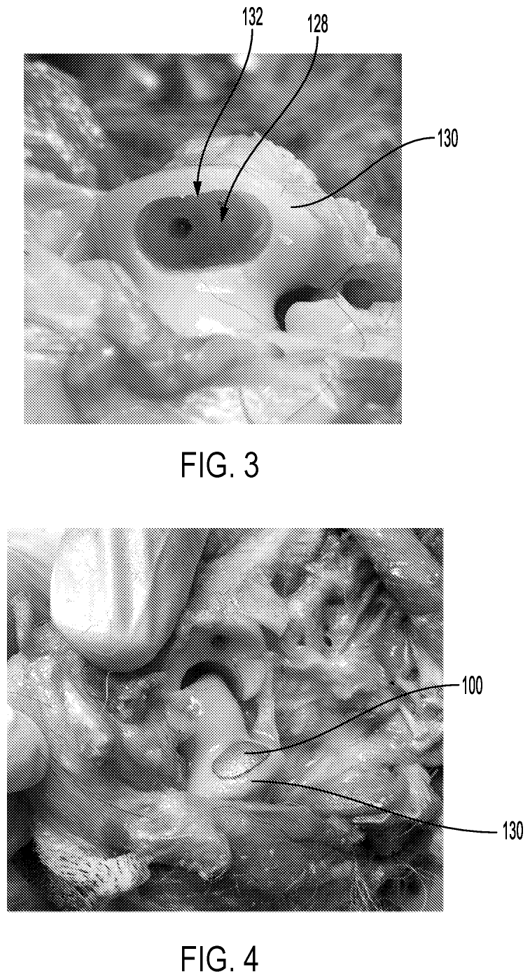

A method includes fixing a humeral implant 100 to a bone of a patient. A method can include forming a humeral trough 128 in a humeral condyle 130. In an example, the humeral trough 128 is formed in the humeral condyle 130 to a depth about equal to a height of the base 102 of the humeral implant 100 such that the articulating surface 110 extends above the height of the humeral trough 128. In an example, the humeral trough 128 is oval in shape. An example humeral trough 128 is shown in FIG. 3. A method can also include inserting the humeral implant 100 into the humeral trough 128. In an example, the humeral implant 100 is press fit into the humeral trough 128. Other fixation methods are possible as well. The humeral implant 100 is shown positioned in the humeral trough 128 in FIG. 4. A method can also include removing a portion of an abaxial rim 132 of the humeral trough 128 to accommodate the outwardly extending second edge 114 of the humeral implant 100.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” or “an example” means that one or more feature, structure, or characteristic described in connection with the example is included in at least one implementation. The phrases “one embodiment” or “one example” or “an example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112 (f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to +5% of a given value.

Illustrative, non-exhaustive examples, which may or may not be claimed, of the subject matter according the present disclosure are provided below.

What is claimed is:

1. A humeral implant comprising:
a base having a first end and a second end opposite the first end, wherein the base includes a sidewall coupling the first end to the second end; and
an articulating surface positioned at the second end of the base, wherein the articulating surface includes a first edge and a second edge opposite the first edge, wherein a radius of curvature of the first edge is greater than a radius of curvature of the second edge, and wherein the second edge of the articulating surface extends away from the sidewall of the base such that a surface area of the articulating surface is greater than a surface area of the first end of the base.

2. The humeral implant of claim 1, wherein the first edge of the articulating surface is aligned with the sidewall of the base.

3. The humeral implant of any one of claims 1-2, wherein an axis perpendicular to a midpoint of the articulating surface is offset from a longitudinal axis of the base in a direction towards the second edge.

4. The humeral implant of claim 3, wherein the offset ranges from about 3 degrees to about 7 degrees.

5. The humeral implant of any one of claims 1-4, wherein a minimum radius of curvature of the first edge ranges from about 1 mm to about 5 mm, and wherein a minimum radius of curvature of the second edge ranges from about 0.01 mm to about 3 mm.

6. The humeral implant of any one of claims 1-5, wherein from a top view the first edge is substantially straight, and wherein from the top view the second edge is curved.

7. The humeral implant of any one of claims 1-6, wherein the articulating surface further includes a first side edge coupling the first edge to the second edge and a second side edge opposite the first side edge and coupling the first edge to the second edge.

8. The humeral implant of claim 7, wherein the first side edge and the second side edge each have a variable radius of curvature that decreases from the first edge to the second edge.

9. The humeral implant of any one of claims 1-8, wherein the base is has two lines of symmetry, and wherein the articulating surface has a single line of symmetry.

10. The humeral implant of any one of claims 1-9, wherein a height of the first edge with respect to the first end of the base is less than a height of the second edge with respect to the first end of the base.

11. The humeral implant of any one of claims 1-10, wherein the first end of the base comprises a first material, and wherein the second end of the base comprises a second material that is different than the first material.

12. The humeral implant of claim 11, wherein the first material is diffusion bonded to the second material.

13. A method comprising:
forming a humeral trough in a humeral condyle; and
inserting the humeral implant of any one of claims 1-12 into the humeral trough.

14. The method of claim 13, wherein the humeral trough is formed in the humeral condyle to a depth about equal to a height of the base of the humeral implant.

15. The method of any one of claims 13-14, wherein the humeral trough is oval in shape.

16. The method of any one of claims 13-15, further comprising:
removing a portion of an abaxial rim of the humeral trough to accommodate the second edge of the humeral implant.
